(19) United States
(12) Patent Application Publication
(10) Pub. No.: US 2010/0222797 A1
(43) Pub. Date: Sep. 2, 2010

(54) APPARATUS FOR CUTTING OUT AND REMOVING TISSUE CYLINDERS FROM A TISSUE, AND USE THEREOF

(75) Inventor: Joachim Reppenthien, Sauerlach (DE)

Correspondence Address: ROTHWELL, FIGG, ERNST & MANBECK, P.C., 1425 K STREET, N.W., SUITE 800, WASHINGTON, DC, 20005, US

(73) Assignee: WISAP Geseelschaft fuer wissenschaffichen Apparatebau MbH, Sauerlach (DE)

(21) Appl. No.: 12/600,754

(22) PCT Filed: May 19, 2008
(86) PCT No.: PCT/EP2008/004001

§ 371 Date: Apr. 1, 2010

(30) Foreign Application Priority Data
May 18, 2007 (DE) 10 2007 023 207.3
Oct. 17, 2007 (DE) 10 2007 049 796.4

(51) Int. Cl. A61B 17/32

(52) U.S. Cl. 606/180

(57) ABSTRACT

The invention relates to an apparatus for cutting out and removing tissue cylinders from a tissue located within a cavity of the body or a joint and/or in or on a wall section thereof. Said apparatus comprises at least two, especially two cutting devices (12, 12′), each of which has a hollow cylindrical base (14, 14′), a distal opening (18, 18′) at a distal end (16, 16′) of the base (14, 14′), and a cutting element (20, 20′) that surrounds the distal opening (18, 18′). The at least two cutting devices (12, 12′) are disposed inside each other and can be rotated relative to one another. The apparatus further comprises at least one driving device (22) for rotating the respective base (14, 14′) of the at least two cutting devices (12, 12′) about the longitudinal axis (24, 24′) of the base (14, 14′). The invention also relates to the use of said apparatus.

The invention relates to a device for excising and removing cylinders of tissue from tissue located within a body cavity or articular cavity and/or in a wall area of same, and also to its use.

Such devices are generally known. Accordingly, such devices are used, together with a trepan or a trepan sleeve, for example in the medical field of gynaecology for the removal of tissue from a uterus, the removal of a complete uterus or the treatment of myoma, etc. For example, such a device is known from EP 0 522 125 B1. Such devices are furthermore used in medicine in many, sometimes, different, other technical fields, such as for surgery in the abdominal cavity, on the stomach and for the excision of gallbladders or appendices. These devices have, however, been shown to be decidedly disadvantageous in practice. Accordingly, all of such devices only comprise one cutting device having a hollow cylindrical main body, a distal opening at a distal end of the main body and a cutting element surrounding the distal opening. The hollow cylindrical main body is designed to rotate for cutting or punching out the cylinder of tissue. Because of such rotation of the hollow cylindrical main body the tissue, from which the cylinder of tissue should be cut or punched out, respectively, is simultaneously deformed or even sometimes brought into a self rotation. For being able to carry out a precise surgical intervention on the tissue, therefore, further separate medical instruments, especially gripping instruments, such as claw grippers, are indispensable to correspondingly deform or to fix the tissue. As a consequence, the application of such devices is generally decidedly complicated, not very gentle for the patient and is time consuming.

The object of the invention is therefore to provide a device for excising and removing cylinders of tissue from tissue, with which the above disadvantages can be prevented, which is furthermore of particularly simple design and is compact and stable, and at the same time enables a simple and fast removal of an excised or stamped out cylinder of tissue from the area of the operation, ensures a markedly easy and gentle, and particularly time-saving, use and also guarantees a substantially improved and increased patient safety while still enabling this device to be advantageously applied.

With regard to the device, this object is technically achieved in a surprisingly simple manner by the features of claim 1.

Accordingly, by means of the design of the device according to the invention for excising and removing cylinders of tissue from tissue located within a body cavity or articular cavity and/or in a wall area of same, which includes at least two, especially two cutting devices each with a hollow cylindrical main body, a distal opening at a distal end of the main body and a cutting element surrounding the distal opening, that are arranged within each other and designed to rotate relative to each other, and at least one drive device for rotating the respective main body of the at least two cutting devices about its longitudinal axis, a simple, compact and stable construction of the overall device can be achieved. Furthermore, the device according to the invention, which can also be called a morcellator, has the essential advantage that the device does not have to be withdrawn through the trepan sleeve together with another instrument, especially a gripping instrument, such as a claw gripper or the like, for deforming or clamping the tissue during the surgical intervention. By means of the design of the device according to the invention having at least two, especially two of such cutting devices rather a deformation and self rotation, respectively, of the tissue, from which a morcellating cylinder of tissue should be cut and stamped out, respectively, is excluded. This results not least in a noticeably easy, very gentle and especially time-saving operation of the device in general. Finally, the device according to the invention enables a substantially improved patient safety to be achieved.

Further advantageous details of the device according to the invention are described in Claims 2 to 41.

Accordingly, it is within the framework of the invention that the inner cutting device according to Claim 2 is coaxially held by the adjacent outer cutting device of the at least two cutting devices.

Advantageously the inner cutting device according to Claim 3 is held with a slight clearance from the adjacent outer cutting device of the at least two cutting devices.

Alternatively, the inner cutting device according to Claim 4 is held with a larger clearance from the adjacent outer cutting device of the at least two cutting devices, with at least one centering and/or guiding element being arranged between the basic bodies of the inner cutting device and the adjacent outer cutting device. By means of the centering and/or guiding element a coaxial alignment of the adjacent outer cutting device of the at least two cutting devices is achieved.

Furthermore, it is within the scope of the invention that the at least two cutting devices in accordance with Claim 5 can be driven in reverse rotation and/or in synchronous rotation relative to each other by means of the drive device. Of markedly great significance in this connection is particularly the inventive design of a reverse rotating drive, relative to each other, of the at least two cutting devices by means of the drive device. Because of the drive reverse rotation, furthermore, the tissue from which the cylinder of tissue is to be excised or stamped is not subject to any deformation or even self-rotation due to the application of the cutting device. By rotation of the at least two, especially two, cutting devices relative to each other, the forces and moments acting on the tissue when excising or stamping out the cylinder of tissue largely cancel each other out.

For this purpose, a gear device, which is housed by a housing, is arranged in an advantageous manner between the at least two cutting devices and the drive device in accordance with Claim 6.

In a further embodiment of the invention, the gear device, corresponding to the measures of Claim 7, has an output drive gearwheel which is assigned to the drive device, and two input drive gearwheels in effective engagement with the one output drive gearwheel, each of which is assigned to one of the at least two cutting devices.

Preferably, the output drive gearwheel and the two input drive gearwheels of the gear device in accordance with Claim 8 are designed as bevel gears.

The features of Claim 9 serve to simplify the design of the device according to the invention. According to this, the two input drive gearwheels, each of which is assigned to one of the at least two cutting devices, is in simultaneous effective engagement with the output drive gearwheel, which is assigned to the drive device. The rotary axes of the two input drive gearwheels coincide. The rotary axes of the two input drive gearwheels are, in contrast, each arranged offset by ±90° relative to the rotary axis of the output drive gearwheel. With a design of this kind, the ability to drive the at least two cutting devices in reverse rotation relative to each other is easily achieved. Furthermore, the drive can be by means of only a single drive device.

Furthermore, it is provided according to the invention that the output drive gearwheel according to Claim 10 is, advantageously, detachably fixed to a supporting element that is rotatably supported on the housing of the gear device. By means of such a two-part design of the output drive gearwheel, it may be possible to make the supporting element, to which not insubstantial forces and/or moments are generally applied by the drive device for the transmission of the rotary motion, of a more wear-resistant material. Also, due to such a two-part design of the output drive gearwheel, a simple, fast and therefore cost-effective replacement of same is possible in the event of wear.

To simplify the assembly and/or repair, the output drive gearwheel and the supporting element are in this connection, in conjunction with Claim 11, preferably detachably fixed to each other by means of a detent or snap-on connection or a threaded connection. Additional security can be provided by pins, etc.

According to Claim 12, the output drive gearwheel or, if fitted, the supporting element is twist-proof connected to the drive device by means of a spigot-shaped transmission element that has a profile which in cross-section is of a rectangular, square, hexagonal or other polygonal, elliptical, trapezoidal, parallelogram-type or semi-circular shape.

Furthermore, it is within the scope of the invention that the two input drive gearwheels according to Claim 13 are each supported by drive bushes, each of which is assigned to one of the at least two cutting devices. By means of such a two-part design, it is also possible to select different materials for the input drive gearwheels and the drive bushes, corresponding to the particular stress. Also, this means that replacements for repair purposes of the input drive gearwheels can be accomplished simply, quickly and cost effectively.

Advantageously, the two input drive gearwheels according to Claim 14 are each attached to the drive bushes by means of a threaded connection or a detent or snap-on connection. Without being shown in detail, a separate securing device, for example by means of pins, etc., can additionally be provided.

According to the measures of Claim 15, the drive bushes advantageously extend through the housing of the gear device, in order to provide a constructive connection between the input drive gearwheel and the at least two assigned cutting devices in each case.

Furthermore, it is within the scope of the invention that the drive device in accordance with Claim 16 can be controlled by a, especially electronic, open-circuit and/or closed-circuit device, in such a way that the at least two cutting devices can each be driven by the drive device at intervals in an opposite drive direction. This ensures that two cutting devices adjacent to each other can be driven in any manner in a right-left rotation and/or in reverse to this and can also be driven in any manner in a left-right rotation.

In this connection, it is advantageous if the time intervals for driving the at least two cutting devices in opposite driving directions in accordance with Claim 17 can be adjusted and/or preset as required by means of the, especially electronic, open-circuit and/or closed-circuit control device.

The at least two cutting devices are each provided, in accordance with the features of Claim 18, with coupling elements, which are unreleasably or undetachably connected to the respective main body, at the proximal end and/or in the area facing the proximal end of the main body.

Of particular interest are the design measures of Claim 19. According to these, the at least two cutting devices and the drive device are twist-proof connected to the gear device by a coupling device in each case. The coupling device in this case is of a design which makes it particularly simple to use by the respective user.

For this purpose, the coupling device according to Claim 20 is designed as plug-in, detent or snap-on device. Such a plug-in, detent or snap-on device is particularly advantageous because during the operative engagement a simple and fast connection of the at least two cutting devices and the drive device with the gear device and subsequent separation of the connection is enabled, including under the circumstances where the user uses only one hand.

In this connection it is particularly advantageous that the coupling device according to Claim 21 has a tilting lever that is arranged on the drive bushes and the housing of the gear device so that it can be swiveled about a rotary axis, is subject to the force of a spring at one end and at an end opposite to the one end has an approximately radially arranged pin, spigot or projection for engaging in a correspondingly shaped hole or recess in the coupling element of the at least two cutting devices or of the drive device.

Furthermore, it is within the scope of the invention that the coupling element of the inner/innermost of the at least two cutting devices according to Claim 22 is designed as a sealing device for closing and releasing a proximal opening at the proximal end of the main body.

In an advantageous manner, the sealing device according to the features of Claim 23 has a two-part, essentially pot-shaped housing with a housing element fitted to the main body and a cover or cap-shaped closing element that can be fitted to the housing element.

The design measures of Claim 24 provide, in a quite advantageous manner, a simple, fast, reliable, and particularly cost-effective manufacture and assembly. Accordingly, the housing element and the closure element of the housing are releasably or unreleasably connected to each other by a threaded connection or a latching or snap-on connection. The constructive design of providing a releasable or unreleasable connection between the housing element and closure element of the housing in this case depends essentially on the type of use and purpose of the device according to the invention, i.e., whether the device is designed to be used once or several times. This in turn, and also the other way round, substantially affects the overall design of the device and the choice of materials of which the individual components of the device according to the invention are made.

If, for example, the device is meant just for single use, i.e., completely disposed of after use, the housing element and the closure element of the housing according to Claim 25 can be unreleasably connected to each other preferably by an adhesive agent, particularly a silicone adhesive, arranged in-between or by means of ultrasonic welding.

For multiple use, the housing element and the closure element of the housing, which are then obviously made of metal, are on the other hand merely screwed together by means of a threaded part.

As an alternative to the threaded connection, a latching or snap-on connection can be provided, by means of which the housing element and the closure element of the housing according to Claim 26 are connected to each other through at least one latching projection or similar latching lug on the housing element and at least one further latching projection or similar latching lug on the closure element, which interact with each other and can be brought into mutual engagement. For a kinematic reversal, it is equally possible to provide the at least one latching projection or similar latching lug on the closure element and the at least one latching recess or similar on the housing element. Instead of the at least one further latching projection, at least one latching recess or similar can equally be provided, which interacts with the at least one latching projection.

For single use, the device according to the invention is in this connection advantageously designed according to the features of Claim 27. Accordingly, the at least one latching projection or similar latching lug on the housing element or on the closure element and the at least one further latching projection or similar latching lug on the closure element or on the housing element are designed in such a way that the housing element and the closure element of the housing are either on one hand unreleasably connected to each other after engagement of the at least one latching projection or similar latching lug and the at least one further latching projection or similar latching lug or on the other hand cannot be connected to each other after the at least one latching projection or similar latching lug and the at least one further latching projection or similar latching lug have been disengaged from each other. With the latter arrangement, it would, for example, be conceivable to provide one of the two latching projections or similar latching lugs with a design breakpoint, so that this latching projection or similar latching lug breaks away from the housing element or from the closure element if a certain tensile force is applied. Instead of the at least one further latching projection, at least one latching recess or similar, which interacts with the at least one latching projection, can equally be provided.

Of decidedly great importance are, furthermore, the design measures of Claim 28, according to which the sealing device includes two sealing elements which are arranged adjacent to each other in the housing on the longitudinal axis of the main body of the cutting device and interact with each other.

Moreover, it is provided according to the invention that one of the two sealing elements of the sealing device according to Claim 29 is essentially designed in the shape of a cone, a truncated cone, a pot, a hat or similar, has a slotted opening that is arranged in a plane which is offset in the direction of the distal end of the main body of the cutting device with respect to a plane extending through the base of the sealing element, and a circumferential section extending radially outwards in the plane that extends through the base of the sealing element, for fixing in the housing. Therefore the one of the two sealing elements is forced into its closed position and held there, due to its shape and the arrangement in the housing, in the opposing direction to the effect of a pressure artificially established within a body cavity or articular cavity, for example due to CO₂ insufflation. The sealing element is thereby pressed from the outside against another instrument or its shaft, for example a gripping or other cutting instrument, especially a claw gripper or myoma drill, to form a complete seal. Accordingly, it is ensured that the pressure, which is necessary for operating, i.e., for excising or punching out a cylinder of tissue from tissue and then removing the excised or punched out cylinder of tissue from the operating area, is maintained in any event. Consequently, an escape of gas, such as CO₂ or similar, from the body cavity or articular cavity is precluded and impedance of the removal of the cylinder of tissue or a traumatisation of the surrounding tissue during the removal of the cylinder of tissue is counteracted.

In particular, it is within the framework of the invention that one of the two sealing elements according to Claim 30 has at least two sealing lips, which are in mutual effective engagement to close and open the slotted opening.

Furthermore, another of the two sealing elements of the sealing device according to Claim 31 is designed in the form of a disc and has a central, approximately circular opening through which a further instrument, especially a gripping instrument or other cutting instrument, preferably a claw gripper or a myoma drill, can be inserted.

The central, approximately, circular, opening of the other of the two sealing elements preferably has, according to Claim 32, an internal diameter that is smaller or equal to an external diameter of the another instrument.

Furthermore, it is within the framework of the invention that the other of the two sealing elements is arranged, according to the design in Claim 33, in the housing along the longitudinal axis of the main body of the cutting device between the proximal end of the main body and the one of the two sealing elements, especially adjacent to the plane extending through the base of the one of the two sealing elements. A mutual interaction of the two sealing elements, whereby the seal can be further improved, is achieved in this way.

To improve the sealing effect the at least one, especially the two, sealing element(s) of the sealing device according to Claim 34 is/are made of flexible plastic, especially silicon.

Of particular interest are the features of Claim 35. According to this, the cutting elements of the at least two cutting devices, which surround the distal opening, are designed as essentially chamfered, tapered, conical or sloping, facing away from the distal opening and running outwards and/or facing to the distal opening and running inwards.

With this respect, the cutting elements of the at least two cutting devices according to Claim 36 advantageously have a very sharp, surrounding and/or continuous or discontinuous smooth, wavy or saw-tooth edge cut which performs a precise cut into the tissue when the main body is rotated, without the remaining tissue being excessively traumatised. The tissue can thus be removed without the surrounding tissue being deformed by the operation.

According to Claim 37, the cutting elements of the at least two cutting devices are preferably fitted with a wavy edge at the foremost edge of the distal opening. This arrangement of the invention results in several circular arc sections distributed around the circumference, with wave valleys axially retreating and wave peaks axially advancing. For example, if there were four wave peaks and four wave valleys retreating, slightly axially in the circumferential area and lying in-between the four wave peaks, circular arc sections in the approximate range of 40 to 45° would result. The wavy edge can in this case be circumferential or continuous, i.e., be present over the complete circumference of the distal opening. Advantageously, the sharp zone in the transition area between the wave valley and wave peak can be designed to be particularly sharp, so that axial fixing of the tissue can be first achieved and then the morcellating of tissue cylinders or tissue sleeves carried out by the rotary movement of the main body. The wavy edge may also, however, be discontinuous in the form of an imbricated arrangement around the casing circumference at the distal opening.

The same applies for the alternative embodiment of the invention according to Claim 38. According to this, the cutting elements of the at least two cutting devices with the saw-tooth edge have teeth distributed over the circumference with axially retreating tooth roots and axially advancing tooth crests. A circumferential and/or continuous or discontinuous saw-tooth edge is conceivable depending on the required application.

For punching out or excising tissue, the rotary movement can be applied to the main body in accordance with Claim 39 by the drive device by means of a motor which can be driven electrically, by means of a battery, pneumatically, or hydraulically.

In a particularly advantageous manner, the housing of the gear and/or the centering and/or guide element and/or the output drive gearwheel and/or the (two) input drive gearwheel(s) and/or the supporting element and/or the drive bushes and/or the coupling elements and/or the sealing device and/or the housing element and/or the closure element of the housing and/or the clamping disc according to Claim 40 can be made of plastic, especially polyoximethylene, polyester, ABS, acrylic, polycarbonate, tetrafluorethylene or Impax, duroplastic elastomers with or without glass fibre reinforcement. The device according to the invention, which in this case is intended for single use, can in this way be manufactured at significant cost saving.

As an alternative to this, it is also within the framework of the invention for the main body of the at least two cutting devices and/or the housing of the gear and/or the centering and/or guide element and/or the output drive gearwheel and/or the (two) input drive gearwheel(s) and/or the supporting element and/or the drive bushes and/or the coupling elements and/or the sealing device and/or the housing element and/or the closure element of the housing and/or the clamping disc according to Claim 41 to be made of metal, especially (stainless) steel, high-grade steel, aluminum, brass, zinc, leaded red brass or an alloy of same. In this case, the device can be returned to multiple use after appropriate thermal and/or chemical sterilisation.

The device can thus be completely sterilized thermally and/or chemically and restored for immediate multiple use.

The designed measures according to Claim 42 are also of particular interest. According to these, the gear device, which is arranged between the at least two cutting devices and the drive device, is designed and can be assembled with its individual components in the form of a module. The gear device and thus the complete device can in this way be assembled as a module by the operator or servicing personnel. On one hand, this enables additional assembly costs to be saved by the manufacturer and, on the other hand, the device according to the invention can thus be simply, quickly and reliably dismantled and then reassembled for further use, e.g., for sterilization or similar.

In this connection, the features of Claim 43 are of great advantage because, according to these, the housing of the gear device or the housing element and the housing cover and/or the output drive gearwheel and/or the one or two input drive gearwheel(s) and/or the supporting element is/are designed and can be assembled in the form of a module.

This object is finally achieved also with regard to use by the features of Claim 44.

Accordingly, a device can be used for excising and removing cylinders of tissue from tissue located within a body cavity or articular cavity and/or in a wall area of same, for example of an organic body at least partially held by and/or surrounded by an organic cavity of a human body, preferably for the removal of embryos, myomas, tumours, ulcers and carcinomas. The device according to the invention is particularly suitable for laparoscopic, thoracoscopic or arthroscopic and similar minimal-invasive surgical procedures. Without being shown in detail, it is also possible with the aid of the device according to the invention to excise and remove inorganic bodies such as gallstones and bladder stones or similar, which are located within a body cavity or articular cavity and/or in a wall area of same, in the form of such body cylinders.

Further features, advantages and details of the invention are given by the following description of some preferred embodiments of the invention and by the aid of drawings. The drawings are as follows:

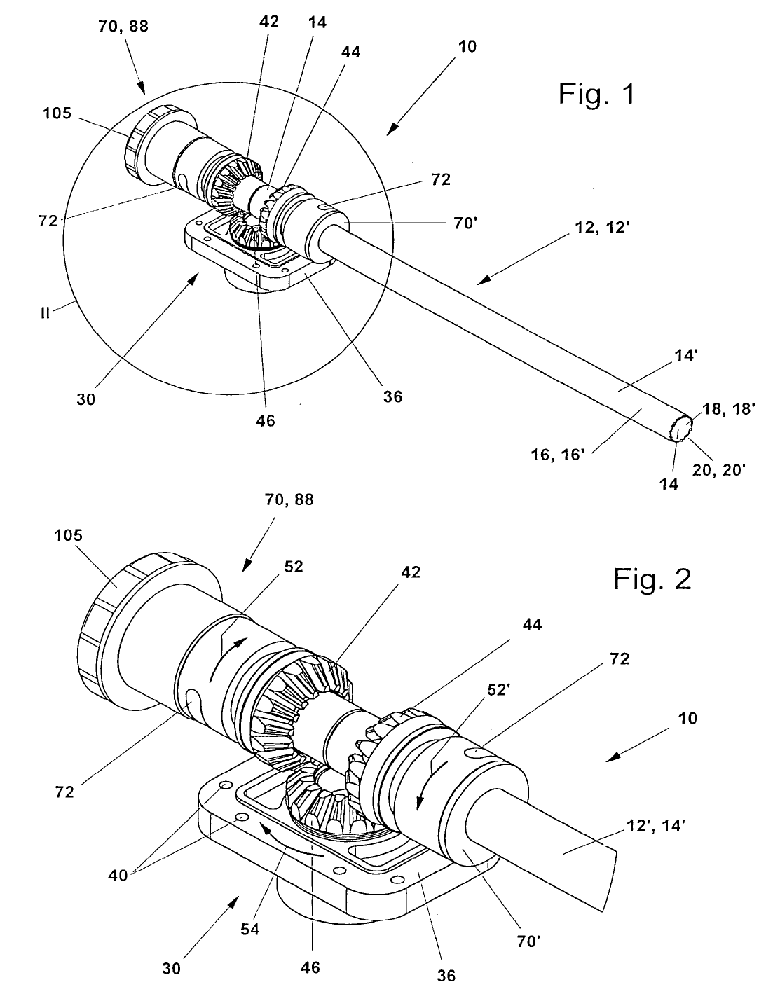

FIG. 1 A perspective front view of an embodiment of a device according to the invention for excising and removing cylinders of tissue from tissue, without a housing.

FIG. 2 A partially cutaway, perspective view of the embodiment of the inventive device according to Section II in the enlarged view in FIG. 1.

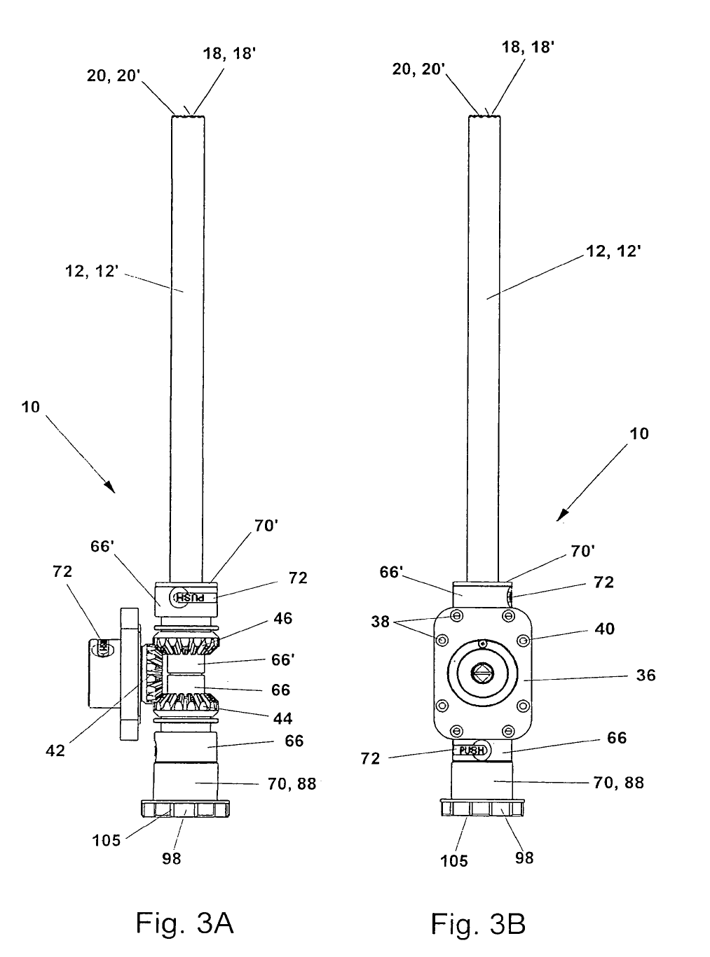

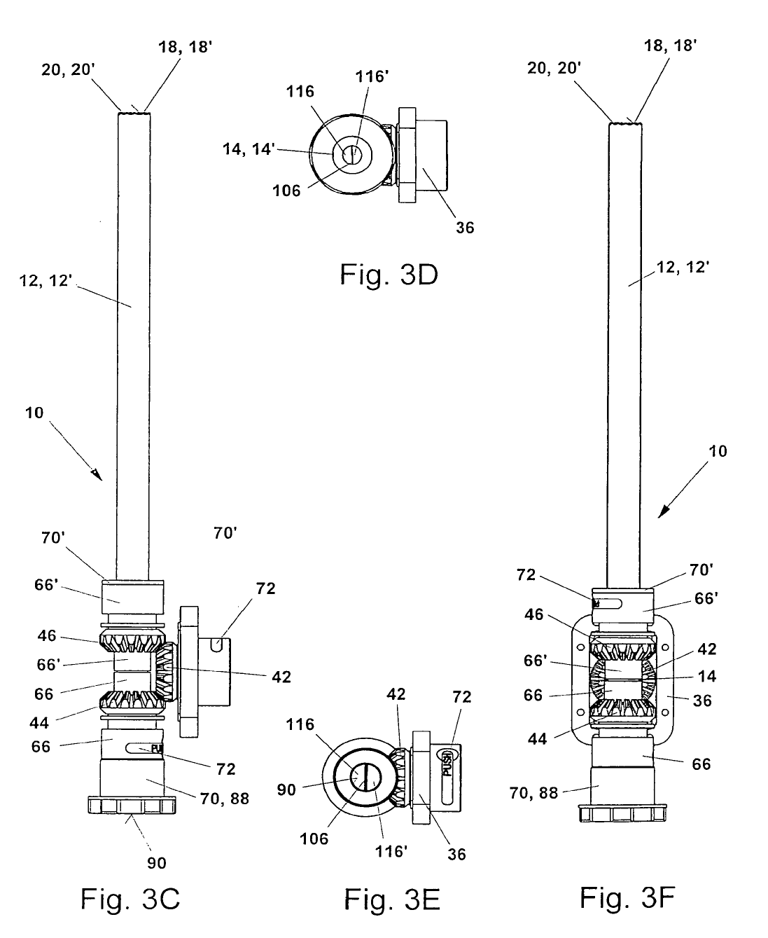

FIG. 3A to 3F A side view, a bottom view, a further side view, a front view, a rear view and a plan view of the embodiment of the inventive device corresponding to FIGS. 1 and 2.

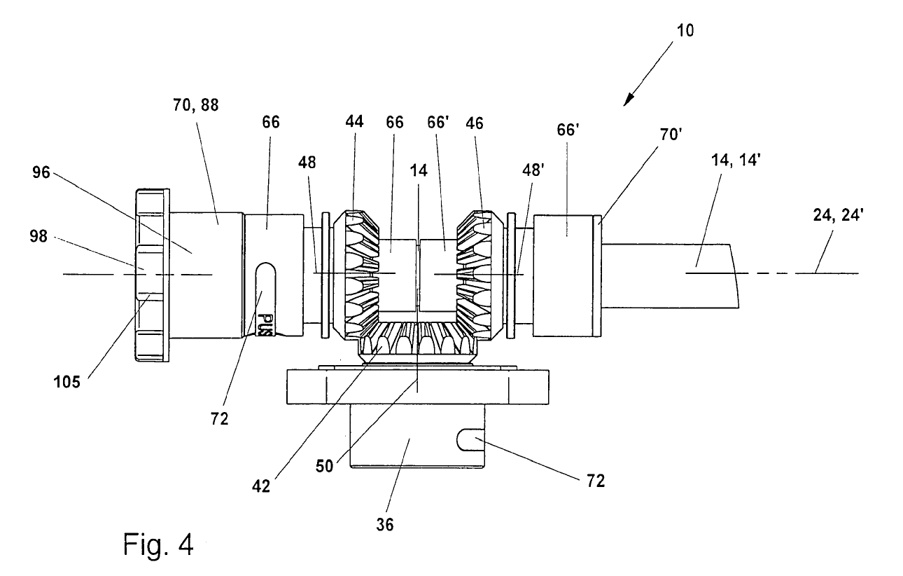

FIG. 4 A partially cutaway side view of the embodiment of the inventive device according to FIG. 3c, shown enlarged.

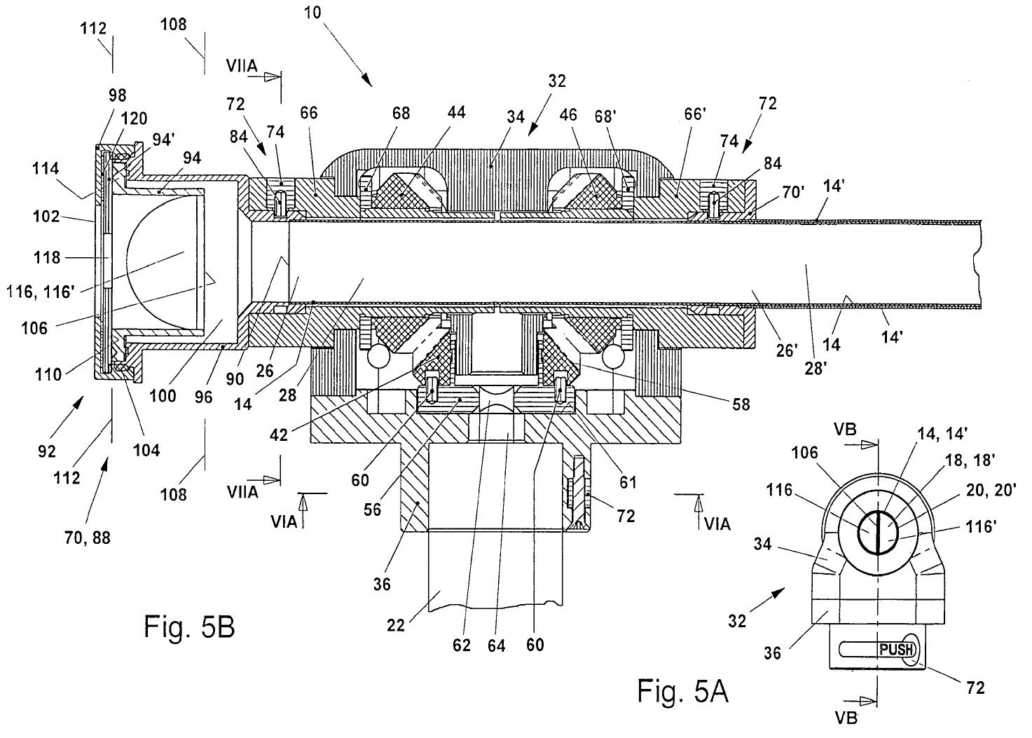

FIGS. 5A und 5B A front view and a cross-section view through the embodiment of the inventive device along the line VB-VB shown in FIG. 5A, with housing, shown enlarged.

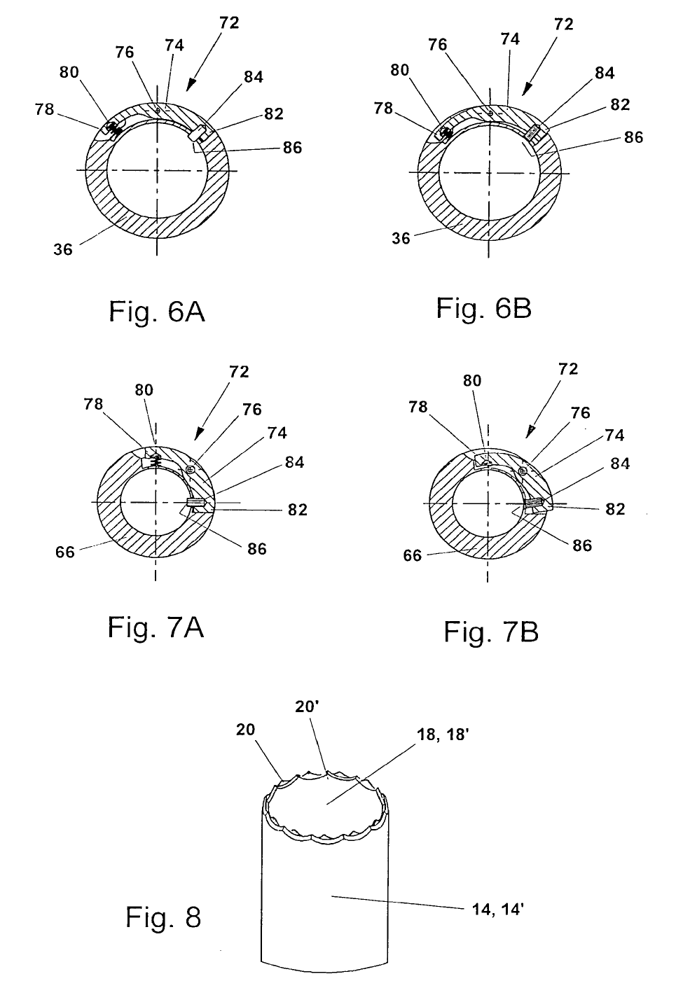

FIGS. 6A und 6B Schematic cross-section views through the embodiment of the inventive device along line VIA-VIA in FIG. 5B, shown in the closed and in the open positions.

FIGS. 7A and 7B Schematic cross-section views through the embodiment of the inventive device along line VIIA-VIIA in FIG. 5B, shown in the closed position and in the open position.

FIG. 8 A cutaway, schematic perspective view of cutting elements of the embodiment of the inventive device according to FIGS. 1 to 7B, shown enlarged.

The device 10 in accordance with the invention is provided for the excising and removal of cylinders of tissue from tissue located within a body cavity or articular cavity and/or in a wall area of same. In the following description of various examples of embodiments of the device 10 in accordance with the invention, components which are the same as each other are given identical reference characters in each case.

The device 10 according to the invention is suitable in its widest sense for operations, especially the removal of organic tissue, preferably of myomas, tumours, ulcers, carcinomas, etc., or an inorganic body, such as gallstones or bladder stones or similar agglomerations, which are at least partially held and/or surrounded by a body cavity or articular cavity or an organic cavity of a human or animal body and/or in a wall area of same.

FIGS. 1 to 7B show an advantageous embodiment of such a device 10 according to the invention.

The device 10 includes at least two cutting devices 12, 12′ each with a hollow cylindrical main body 14, 14′. A distal opening 18, 18′ is, in each case, located at the distal end 16, 16′ of the main body 14, 14′. Each distal opening 18, 18′ is surrounded by a cutting element 20, 20′.

With the device shown in FIGS. 1 to 7B, two such cutting devices 12, 12′ are provided, which corresponds to a particularly advantageous realization of the invention. Without being shown in detail, it is, however, equally possible in principle to provide three or even more cutting devices 12, 12′.

The two cutting devices 12 and 12′ in this case are arranged inside each other. The inner cutting element 20 is thus housed by the outer cutting element 20′. Furthermore, the two cutting elements 20, 20′ are designed to be rotatable relative to each other.

The inner cutting device 12 is thus coaxially housed by the adjacent outer cutting device 12′ of the two cutting devices 12, 12′. A slight clearance is preferably provided between the inner cutting device 12 and the adjacent outer cutting device 12′. Consequently, the outer diameter of the main body 14 of the cutting device 12 is slightly smaller than the internal diameter of the main body 14′ of the cutting device 12′, and vice versa. The clearance between the inner cutting device 12 and the adjacent outer cutting device 12′ should in this case be chosen in such a way that the cutting devices 12, 12′ can be rotated easily relative to each other but at the same time enable a precise guidance relative to each other or between each other. Furthermore, the passage of blood, bodily fluid or gas, etc., between the outer diameter of the main body 14 of the cutting device 12 and the inner diameter of the main body 14′ of the cutting device 12′ should be, if possible, completely or largely prevented.

Without being shown in detail, it is possible as an alternative for the inner cutting device 12 to also be housed by the adjacent outer cutting device 12′ with a greater clearance. However, with such an embodiment, one or more centering and/or guide element(s) (not illustrated) is/are arranged between the basic bodies 14, 14′ of the inner cutting device 12 of the adjacent outer cutting device 12′. This/these centering and/or guide element(s) should be designed particularly to prevent tilting and/or sluggishness during rotation of the inner cutting device 12 relative to the adjacent outer cutting device 12′, and also prevent the passage of blood, bodily fluid or gas, etc.

For rotation of the particular main body 14, 14′ of the two cutting devices 12, 12′ about their respective longitudinal axis 24, 24′, the device 10 is provided with at least one drive device 22. With the exemplary embodiment of the device 10 shown, only one such drive device 22 is provided (shown schematically). The drive device 22 can be arranged at a proximal end 26 of one of the basic bodies 14, 14′. Alternatively or, as shown in this present exemplary embodiment, the drive device 22 is also arranged in an area 28 of the main body 14 which is simply facing towards and/or adjacent to the proximal end 26. The drive device 22 is designed as a motor, which, for example, can be driven electrically, by means of a battery, pneumatically, or hydraulically.

In a particularly advantageous manner, the two cutting devices 12, 12′ can be driven in reverse rotation relative to each other by the drive device 22. In this way, it is furthermore possible, in a particularly simple and thus cost-effective manner, for the tissue (not illustrated) from which the relevant cylinder of tissue is to be excised and/or stamped out to be neither deformed nor caused to rotate within the body cavity or articular cavity and/or in or at the wall area of same during the surgical intervention by means of the device 10 according to the invention. Due to the reverse rotation of the two cutting devices 12, 12′, the forces and/or any moments acting on the tissue due to the relevant cutting elements 20, 20′ in any case cancel each other out. The result of this is that the tissue remains undeformed or in a specific position without moving, during the surgical intervention using the device 10 in accordance with the invention.

Without being shown in detail, the drive device 22 can be controlled by an, especially electronic, open-loop and/or closed-loop control device (not illustrated) so that the two cutting devices 12, 12′ can each be driven at intervals in an opposite drive direction by the drive device 22. The change in the drive direction of the two cutting devices 12, 12′, which is of an oscillating or similar alternating nature, is independent of their rotary movement relative to each other. In other words, the drive of the two cutting devices 12, 12′ can take place in an opposite drive direction without their drive relative to each other in reverse rotation and/or in synchronous rotation being cancelled out. In this way it is ensured that two particular adjacent cutting devices 12, 12′ can be driven in any manner in a right-left rotation and/or in the reverse to this and also in any way in a left-right rotation.

Thereby it is of particular advantage if the time intervals for driving the two cutting devices 12, 12′ in an opposite drive direction can be set and/or preset as required by the, especially electronic, open-circuit and/or closed-circuit control device. In this connection, time intervals which are equal or of different length can be chosen, precisely matched to the particular surgical area in question, or can be randomly chosen, etc.

For operating in reverse rotation and/or a synchronous rotation of the two cutting devices 12, 12′ relative to each other by means of the drive device 22, a gear device 30, which is fitted in a housing 32, is arranged between the two cutting devices 12, 12′ and the drive device 22. The housing 32 in this case consists of a housing element 34, which is shown only in FIGS. 5A and 5B, and a housing cover 36 which is shown in all the illustrations, FIGS. 1 to 7B. The housing element 34 and the housing cover 36 can be secured to each other in a conventional manner by screws 38, which pass through corresponding holes or recesses in the housing element 34 (not illustrated) and corresponding holes 40 or recesses in the housing cover 36.

For operating in reverse rotation, the gear device 30 has an output drive gearwheel 42 which is assigned to the drive device 22. Furthermore, the gear device 30 has two input drive gearwheels 44, 46 which are in an effective engagement with the output drive gearwheel 42 and each of which is assigned to one of the two cutting devices 12, 12′.

The output drive gearwheel 42 and the two input drive gearwheels 44, 46 of the gear device 30 are, in the embodiment of the inventive device 10 shown in FIGS. 1 to 7B, each designed as bevel gears.

In the embodiment of the device 10 shown in FIGS. 1 to 7B, the two input drive gearwheels 44 or 46, each of which is assigned to one of the two cutting devices 12 or 12′, are in simultaneous effective engagement with the output drive gearwheel 42, which is assigned to the drive device 14. With the present embodiment, the rotary axes 48, 48′ of the two input drive gearwheels 44, 46 coincide. The rotary axes 48, 48′ of the two input drive gearwheels 44, 46 are furthermore identical to the longitudinal axes 24, 24′ of the basic bodies 14, 14′. In contrast, the rotary axes 48, 48′ of the two input drive gearwheels 44, 46 are arranged offset once by +90° and once by −90° relative to the rotary axis 50 of the output drive gearwheel 42. By a design feature of this kind, a simultaneous, uniform rotation, which is also in the opposite direction, of the two input drive gearwheels 44, 46 corresponding to the arrows 52, 52′ is achieved by rotating the output drive gearwheel 42 corresponding to arrow 54. If, on the other hand, the output drive gearwheel 42 rotates in the opposite direction to the arrow 54, the direction of rotation of both input drive gearwheels 44, 46 also reverses corresponding to the arrows 52, 52′ (not illustrated).

As particularly shown in FIGS. 2 and 5B, the output drive gearwheel 42 is furthermore detachably fixed to an approximately disc-shaped supporting element 56. For this purpose, the output drive gearwheel 44 and the supporting element 56 are detachable fixed to each other by means of a detent or snap-on connection 58. Furthermore, pins 60, screws or similar, are provided in order to prevent relative movement between the output drive gearwheel 42 and the supporting element 56.

The disc-shaped supporting element 56 is rotatably supported on the housing cover 36 of the housing 32 of the gear device 30. For example, the supporting element 56 is mounted in a round recess 61. The supporting element 56 is, furthermore, provided with a centrally arranged recess 62 in which a spigot-shaped transmission element 64 is in twist-proof engagement. The centrally arranged recess 62 and the transmission element 64 are formed to correspond to each other. The transmission element 64 can thus, for example, be provided with a profile which in cross-section is rectangular, square, hexagonal, or polygonal, elliptical, trapezoidal, of parallelogram type or semi-circular shape.

The two-part design of the output drive wheel 42 and supporting element 56 on one hand enables various materials with different properties to be used. For example, the output drive gearwheel 42 in the present exemplary embodiment of the device is made of polyoximethylene (POM), whereas the supporting element 56 consists of polyphenylenesulfone (PPSU). As is generally known, where high forces and/or moments occur such as exerted by the drive device 22 via the transmission element 64 to the supporting element 56, polyphenylenesulfone is far more resistant and wear-proof than many other plastics. On the other hand, a design of this kind enables a simple, fast and therefore cost-effective replacement of the output drive gearwheel 42 in the event of wear or other repair.

The same applies for the two input drive gearwheels 44, 46 which in the present embodiment of the device 10, which is shown in FIGS. 1 to 7B, are also of two-part design. Accordingly, the two input drive gearwheels 44, 46 are each supported by drive bushes 66, 66′, each of which is assigned to one of the two cutting devices 12, 12′.

According to FIG. 5B, the two input drive gearwheels 44, 46 in the device 10 shown are each secured to the drive bushes 66, 66′ by a threaded connection. For locking, washers (flat or thrust) 68, 68′ are provided, against which the input drive gearwheels 44, 46 can be locked. In addition, it is conceivable to secure the input drive gearwheels 44, 46 by means of pins or screws (not illustrated) or by an adhesive, preferably a silicone adhesive, placed in-between, or by means of ultrasound welding but an additional attachment of this kind might make a fast assembly or dismantling for repair purposes difficult. The drive bushes 66, 66′ themselves extend, as can be clearly seen in FIG. 5B, through the housing 32 of the gear device 30.

The at least two cutting devices 12, 12′, as can also be seen from FIG. 5B, are provided with coupling elements 70, 70′, which are undetachably connected to the respective main body 14, 14′ of the cutting devices 12, 12′. The coupling elements 70, 70′ can in this case be arranged at the proximal end 26, 26′. Alternatively or cumulatively, the coupling elements 70, 70′ are, as shown in the exemplary embodiment, arranged in an area 28, 28′ of the main body 14, 14′ which is facing towards the proximal end 26, 26′.

The two cutting devices 12, 12′ and the drive device 22 can also have a twist-proof connection to the gear device 30, by means of a coupling device 72 in each case. The coupling device 72 is designed as a plug-in, detent or snap-on device.

For this purpose, the coupling device 72 has a tilting lever 74. The tilting lever 74 is arranged on the drive bushes 66, 66′ and the housing 32 of the gear device 30 so that it can be rotated about a rotary axis 76. One end 78, the tilting lever is subject to the force of a spring 80. At one end 82, which lies opposite the end 78, a pin 84, spigot or projection is provided, which interacts with a correspondingly shaped hole 86 or recess on the assigned coupling element 70, 70′ of the two cutting devices 12, 12′ or of the drive device 22 (not illustrated). The pin 84, spigot or projection and the associated hole 86 or recess runs, approximately radially, relative to the longitudinal axes 24, 24′ of the basic bodies 14, 14′ of the device 10.

The spring 80 is designed in such a way that the tilting lever 74 is permanently held in the closed position of the coupling device 72. In the closed position, which is shown in FIGS. 6A and 7A, the radially arranged pin 84, spigot or projection is in engagement with the hole 86 or recess. The particular cutting device 12, 12′ or the drive device 22 is thus twist-proof connected to the gear device 30.

In the open position of the coupling device 72, on the other hand, which is achieved by pressing the tilting lever 64 down against the force of the spring 80, the tilting lever 74 with the pin 84, spigot or projection is swung radially outwards. The pin 84, spigot or projection is therefore, as a consequence, disengaged from the hole 86 or recess in the open position, which is shown in FIGS. 6B and 7B. The cutting devices 12, 12′ or the drive device 22 can thus be easily separated from the gear device 30.

With the embodiment of the device 10 according to the invention, which is shown in FIGS. 1 to 7B, the coupling element 70, which is assigned to the inner cutting device 12 of the two cutting devices 12, 12′, is designed as sealing device 88.

The sealing device 88 for closing and opening a proximal opening 90, which is located at the proximal end 26 of the main body 14. The sealing device 88 includes a housing 92 and at least one sealing element 94, 94′ which can be accommodated in the housing 92. As shown in FIGS. 1 to 7B, a total of two sealing elements 94, 94′ are provided on the illustrated embodiment of the device 10 in accordance with the invention. Without being shown in detail, it is easily conceivable to merely provide a single sealing element, for example as an integral component of both illustrated sealing elements 94, 94′, or also more than two such sealing elements 94, 94′.

The housing 92 is formed from a housing element 96 and a closure element 98.

The housing element 96 is essentially pot-shaped, accommodates the at least one sealing element 94, 94′ and is unreleasable attached to the main body 14. The at least one sealing element 94, 94′ extends at least in part into a chamber 100 which is enclosed by, and therefore formed by, the housing element 96 of the housing 92.

The closure element 98 is cover- or cap-shaped. The closure element 98 is provided with a central, approximately circular bore 102 or similar opening. A further instrument (not illustrated), especially a gripping or other cutting instrument, preferably a claw gripper or myoma drill, can be passed through the bore 102, i.e., via the bore 102 through the proximal opening 90 of the main body 14, through the sealing device 88 at the proximal end 26 of the main body 14, through the main body 14 itself on to the distal end 16 of the main body 14, through the distal opening 18 finally to the (cylinder of) tissue to be removed in the operating area. The closure element 98 can in turn be attached to the housing element 96.

For this purpose, the housing element 96 and the closure element 98 of the housing 92 can be connected to each other by a threaded connection or a latching or snap-on connection 104, and the connection can be either releasable or unreleasable. Whether it is designed with a releasable or unreleasable connection between the housing element 96 and the closure element depends on the use and purpose of the device 10, i.e., whether it is intended for single (one-off) use or multiple use, the construction of the device 10 and the choice of materials from which the individual components of the device 10 in accordance with the invention are made, the wishes of the users, etc.

In the case of a single use of the device 10, the housing element 96 and the closure element 98 are unreleasable connected to each other by a threaded connection or a latching or snap-on connection 104 with an adhesive agent, preferably a silicone adhesive, arranged in between, or by means of ultrasonic welding. In this way, a thermal or chemical sterilisation of the device 10 and any repeated or further use of the device 10 are ruled out.

The housing 92 of the sealing device 88 and especially the closing element 98 of the housing 92 is provided on its circumference with a profile 105 for the manual operation and rotation of the main body 14.

With the embodiment shown in FIGS. 1A to 7B, the sealing device includes, as already mentioned, two sealing elements 94, 94′, which are arranged adjacent to each other in the housing 92 on the longitudinal axis 24 of the main body 14 of the cutting device 12 and interact with each other.

One 94 of the two sealing elements 94, 94′ of the sealing device 88 is essentially designed in the shape of a cone, a truncated cone, a pot, a hat or similar. The sealing element 94 has a slotted opening 106 that is arranged in a plane 108 (vertical to the sheet plane), which is offset in the direction of the distal end 16 of the main body 14 with respect to a plane 112 (vertical to the sheet plane) extending to the base 110 of the sealing element 36. The sealing element 94 therefore extends with the part or section essentially designed in the shape of a cone, a truncated cone, a pot, a hat or similar, in the direction of the distal end 16 of the main body 14 and projects into the chamber 100 of the housing 92. Furthermore, the sealing element 94 has a circumferential section 110 extending radially outwards in the plane 112 that extends through the base 110 of the sealing element 94 and serves for fixing in the housing 92.

The sealing element 94 has at least two sealing lips 116, 116′ that are in mutual effective engagement, to close and open the slotted opening 106. Due to the shape and arrangement of the two sealing lips 116, 116′ of the sealing element 36 in the housing 92 in an opposing direction to the action of a pressure artificially created inside a body cavity or articular cavity, for example due to CO₂ insufflation, the sealing lips 116, 116′ seat tightly on the outside against a further instrument (not illustrated), for example a gripping or other cutting instrument, especially a claw gripper or a myoma drill, and form a complete seal. In this way, a virtual wedging of the sealing lips 116, 116′ takes place. The sealing lips 116, 116′ are pressed against the further instrument and remain in this position until there is a controlled release of the pressure. In this way, it is guaranteed that the pressure which is necessary for operating, i.e., excising or punching out a cylinder of tissue from tissue and then removing the excised or punched out cylinder of tissue from the operating area, is permanently maintained. An escape of gas such as CO₂ or similar from the body cavity or articular cavity of the operating area is effectively prevented.

Another 94′ of the two sealing elements 94, 94′ of the sealing device 88 is designed in the form of a disc and is provided with a central, approximately circular opening 118. Said further instrument (not illustrated), especially a gripping or other cutting instrument, preferably a claw gripper or myoma borer, can be inserted through the opening 118 of the sealing element 94′. The central, approximately circular opening 118 is provided with an internal diameter which is smaller or equal to an external diameter of the further instrument.

As can be seen from FIG. 5B, the other sealing element 94′ is arranged in the housing 92 on the longitudinal axis 24 of the main body 14 between the proximal end 26 of the main body 14 and the one sealing element 94. In particular, the other sealing element 94′ is arranged (immediately) adjacent to the plane 112 extending to the base 110 of the one sealing element 94.

The two sealing elements 94, 94′ of the sealing device 88 can be fixed to each other by a clamping disc 120 or similar in the housing 92.

Corresponding to FIGS. 1 and 8, the cutting elements 20, 20′ of the cutting devices 12, 12′ are essentially chamfered, tapered, conical or sloping and facing outwards away from the distal opening 18, 18′ and/or running inwards towards the distal opening 18. With the embodiment of the device 10 shown in FIG. 8, the cutting element 20 of the cutting device 12 is essentially chamfered, tapered, conical or running obliquely inward facing the distal opening 18. In contrast, the cutting element 20′ of the cutting device 12′ is arranged so as to be essentially chamfered, tapered, conical or running obliquely outwards from the distal opening 18′. Without being shown in detail, any other constructive designs or arrangements of the cutting elements 20, 20′ with respect to each other are equally possible.

With this respect, the cutting elements 20, 20′ in each case can have a circumferential or discontinuous smooth edge, wavy edge or toothed edge. With the embodiment of the device 10, which is particularly shown in FIG. 8, both cutting elements 20, 20′ are provided with a toothed edge which has teeth distributed over the circumference with axially retreating teeth roots and axially advancing teeth points. Without being shown in detail, other combinations are also realizable in this respect. Thus, the cutting elements 20, 20′ of the two cutting devices 12, 12′ can, for example, be ground with a wavy edge with axially retreating wave valleys and axially advancing wave peaks over circular arc sections distributed around the circumference.

The at least one, especially the two, sealing element(s) 94, 94′ of the sealing device 88 is/are made of flexible plastic, especially silicone.

The housing 32 of the gear device 30 and/or the centering and/or guiding element and/or the output drive gearwheel 42 and/or the one or two input drive gearwheel(s) 44, 46 and/or the supporting element 56 and/or the drive bushes 66, 66′ and/or the coupling elements 70, 70′ and/or the sealing device 88 and/or housing element 96 and/or the closing element 98 of the housing 92 and/or the clamping disc 120 is/are made of plastic, especially polyoxymethylene, polyester, polyphenylenesulfone, ABS, acrylic, polycarbonate, tetrafluoroethylene or Impax, duroplastic elastomers, with or without glass fibre reinforcement, or a combination of these.

Instead of these, the housing 32 of the gear device 30 and/or the centering and/or guiding element and/or the output drive gearwheel 42 and/or the one or two input drive gearwheel(s) 44, 46 and/or the supporting element 56 and/or the drive bushes 66, 66′ and/or the coupling elements 70, 70′ and/or the sealing device 88 and/or the housing element 96 and/or the closing element 98 of the housing 92 and/or the clamping disc 120 can alternatively and/or cumulatively be made of metal, especially (stainless) steel, high-grade steel, aluminum, brass, zinc, leaded red brass or an alloy of these. In contrast, the basic bodies 14, 14′ of the cutting devices 12, 12′ and/or the coupling device 72 are generally preferably made of metal, especially (stainless) steel, high-grade steel, aluminum, brass, zinc, leaded red bass or an alloy of these.

Without being shown in detail, the gear device 30, which is arranged between the at least two cutting devices 12, 12′ and the drive device 22, can be formed and be capable of assembly as a module with its individual components. The gear device 30 and therefore the complete device 10 can in this way be assembled as a module by the operator or servicing personnel. On one hand, additional assembly costs by the manufacturer can thus be spared and, on the other hand, the device according to the invention can therefore be dismantled and then reassembled for reuse easily, quickly and reliably, for instance for the purpose of sterilization or similar. An advantage in this case is that the housing 32 of the gear device 30 or the housing element 34 and the housing cover 36 and/or the output drive gearwheel 42 and/or the one or two input drive gearwheel(s) 44, 46 and/or the supporting element 56 is/are designed and can be assembled as a module.

The invention is not limited to the embodiments of the device shown in accordance with FIGS. 1 to 8. Accordingly, it is possible without difficulty to not drive, or not drive exclusively, the at least two cutting devices 12, 12′ of the exemplary embodiment of the inventive device 10 shown, in reverse rotation, but instead alternatively or cumulatively in synchronous rotation, provided an operative intervention would make such a corrected drive necessary. Without being shown in detail, additional constructive measures would be generally known, with respect to the gear device 30, for the provision of switching possibilities for the gear device 30, etc. Furthermore, instead of a threaded connection, a detent or snap-on connection (not shown in detail) for the mutual attachment of the housing element 96 and closing element 98 can be provided. In this case, the housing element 96 and closing element 98 are connected together by means of at least one detent projection or similar detent lug on the housing element 96 and/or on the closing element 98 and at least one further detent projection on the closing element 98 and/or on the housing element 96. Both detent projections or similar detent lugs interact with each other and can be brought into mutual engagement. The at least one detent projection or similar detent lug and/or the at least one further detent projection or similar detent lug can preferably in this connection be designed in such a way that the housing element 96 and closing element 98 of the housing 92 are undetachably connected to each other after engagement or, after subsequent disengagement, cannot be reconnected to each other. For example, one or both of the detent projections or similar detent lug(s) can be provided with a break point, in order to permanently damage the device 10 after being disengaged and therefore make it unfit for further use. The device 10 according to the invention is equally well suited to one-off or multiple use.

1. Device for excising and removing cylinders of tissue from tissue within a body cavity or articular cavity and/or in the wall area of same, that includes at least two, especially two cutting devices (12, 12′) each with a hollow cylindrical main body (14, 14′), a distal opening (18, 18′) at a distal end (16, 16′) of the main body (14, 14′) and a cutting element (20, 20′) surrounding the distal opening (18, 18′), that are arranged within each other and designed to rotate relative to each other, and at least one drive device (22) for rotating the respective main body (14, 14′) of the at least two cutting devices (12, 12′) about its longitudinal axis (24, 24′).

2. Device according to claim 1, characterized in that the inner cutting device (12) is coaxially held by the adjacent outer cutting device (12′) of the at least two cutting devices (12, 12′).

3. Device according to claim 1 or 2, characterized in that the inner cutting device (12) is held with a slight clearance from the adjacent outer cutting device (12′) of the at least two cutting devices (12, 12′).

4. Device according to one of claims 1 to 3, characterized in that the inner cutting device (12) is held with a larger clearance from the adjacent outer cutting device (12′) of the at least two cutting devices (12, 12′), with at least one centering and/or guiding element being arranged between the basic bodies (14, 14′) of the inner cutting device (12) and the adjacent outer cutting device (12′).

5. Device according to one of claims 1 to 4, characterized in that the at least two cutting devices (12, 12′) can be driven in reverse rotation and/or in synchronous rotation relative to each other by the drive device (22).

6. Device according to one of claims 1 to 5, characterized in that a gear device (30) held by a housing (32) is arranged between the at least two cutting devices (12, 12′) and the drive device (22).

7. Device according to one of claims 1 to 6, characterized in that the gear device (30) includes an output drive gearwheel (42) that is assigned to the drive device (22) and two input drive gearwheels (44, 46) that are in effective engagement with the one output drive gearwheel (42) and in each case assigned to one of the at least two cutting devices (12, 12′).

8. Device according to one of claims 1 to 7, characterized in that the output drive gearwheel (42) and the two input drive gearwheels (44, 46) of the gear device (30) are designed as bevel gears.

9. Device according to one of claims 1 to 8, characterized in that the two input drive gearwheels (44, 46), which are in each case assigned to one of the at least two cutting devices (12, 12′), are in simultaneous effective engagement with the output drive gearwheel (42) which is assigned to the drive device (22), with the rotary axes (48, 48′) of the two input drive gearwheels (44, 46) coinciding and the rotary axes (48, 48′) of the two input drive gearwheels (44, 46) being arranged, offset±90° in each case, opposite the rotary axis (50) of the output drive gearwheel (42).

10. Device according to one of claims 1 to 9, characterized in that the output drive gearwheel (42) is detachably fixed to a supporting element (56) that is rotatably supported on the housing (32) of the gear device (30).

11. Device according to one of claims 1 to 10, characterized in that the output drive gearwheel (44) and the supporting element (56) are detachably fixed to each other by means of a latching or snap-on connection or a threaded connection.

12. Device according to one of claims 1 to 11, characterized in that the output drive gearwheel (42), especially the supporting element (56), is twist-proof connected to the drive device (22) by means of a spigot-shaped transmission element (64) that has a profile which in cross-section is rectangular, square, hexagonal, or other polygonal, elliptical, trapezoidal, parallelogram type or semi-circular shape.

13. Device according to one of claims 1 to 12, characterized in that the two input drive gearwheels (44, 46) are each supported by drive bushes (66, 66′), each of which is assigned to one of the at least two cutting devices (12, 12′).

14. Device according to one of claims 1 to 13, characterized in that the two input drive gearwheels (44, 46) are each attached to the drive bushes (66, 66′) by means of a threaded connection or a latching or snap-on connection.

15. Device according to one of claims 1 to 14, characterized in that the drive bushes (66, 66′) extend through the housing (32) of the gear device (30).

16. Device according to one of claims 1 to 15, characterized in that the drive device (22) can be controlled (closed-circuit control or open-circuit control) by a, especially electronic, controller in such a way that the at least two cutting devices (12, 12′) can be driven by the drive device (22) at intervals in each case in the opposite drive direction.

17. Device according to one of claims 1 to 16, characterized in that the time intervals for driving the at least two cutting devices (12, 12′) in an opposite drive direction can be set and/or preset as required by a, especially electronic, controller.

18. Device according to one of claims 1 to 17, characterized in that the at least two cutting devices (12, 12′) are each provided with coupling elements (70, 70′), which are undetachably connected to the respective main body (14, 14′), at the proximal end (26, 26′) and/or at the area (28, 28′) of the main body (14, 14′) facing towards the proximal end (26, 26′).

19. Device according to one of claims 1 to 18, characterized in that the at least two cutting devices (12, 12′) and the drive device (22) are twist-proof connected to the gear device (30) by a coupling device (72) in each case.

20. Device according to one of claims 1 to 19, characterized in that the coupling device (72) is designed as a plug-in, detent or snap-on device.

21. Device according to one of claims 1 to 20, characterized in that the coupling device (72) has a tilting lever (74), that is arranged on the drive bushes (66, 66′) and the housing (32) of the gear device (30) so that it can be swiveled about a rotary axis (76), is subject to the force of a spring (80) at one end (78) and at one end (82) opposite to the one end has an approximately radially arranged pin (84), spigot or projection for engaging in a corresponding-shaped bore (86) or recess in the coupling element (70, 70′) of the at least two cutting devices (12, 12′) or of the drive device (22).

22. Device according to one of claims 1 to 21, characterized in that the coupling element (70) of the inner/innermost of the at least two cutting devices (12, 12′) is designed as a sealing device (88) for closing and releasing a proximal opening at the proximal end (26) of the main body (14).

23. Device according to one of claims 1 to 22, characterized in that the sealing device (88) has a two-part, essentially pot-shaped, housing (92) with a housing element (96) fitted to the main body (14) and a cover or cap-shaped closing element (98) that can be fitted to the housing element (96).

24. Device according to one of claims 1 to 23, characterized in that the housing element (96) and the closing element (98) of the housing (92) are connected to each other, detachably or undetachably, by means of a threaded connection or a latching or snap-on connection (104).

25. Device according to one of claims 1 to 24, characterized in that the housing element (96) and the closing element (98) of the housing (92) are can be unreleasably connected to each other preferably by an adhesive, in particular a silicon adhesive, placed in-between, or by means of ultrasonic welding.

26. Device according to one of claims 1 to 25, characterized in that the housing element (96) and the closing element (98) of the housing (92) are connected to each other by at least one detent projection or similar detent lug on the housing element (96) or on the closing element (98) and at least one other detent projection or similar detent lug and/or at least one detent recess or similar on the closing element (98) or on the housing element (96), that interact with each other and can be brought into mutual engagement.

27. Device according to one of claims 1 to 26, characterized in that the at least one detent projection or similar detent lug on the housing element (96) or on the closing element (98) and the at least one other detent projection or similar detent lug and/or the at least one detent recess or similar on the closing element (98) or on the housing element (96) is/are designed in such a way that the housing element (96) and the closing element (98) of the housing (92) are undetachably connected to each other after engagement of the at least one detent projection or similar detent lug and the at least one other detent projection or similar detent lug and/or the at least one detent recess, or after subsequent disengagement of the at least one detent projection or similar detent lug and the at least one other detent projection or similar detent lug and/or the at least one detent recess cannot be connected to each other.

28. Device according to one of claims 1 to 27, characterized in that the sealing device (88) has two sealing elements (94, 94′) that are arranged adjacent to each other in the housing (92) on the longitudinal axis (24) of the main body (14) of the cutting device (12), and interact with each other.

29. Device according to one of claims 1 to 28, characterized in that one (94) of the two sealing elements (94, 94′) of the sealing device (88) is essentially formed in the shape of a cone, truncated cone, pot, hat or similar, has a slot opening (106) that is in a plane (108) that is arranged relative to a plane (112) extending through the base surface (110) of the sealing element (94) in the direction of the distal end (16) of the main body (14) of the cutting device (12) and has a circumferential section (114), extending radially outwards in the plane (112) extending through the base surface (110) of the sealing element (94), for fixing in the housing (92).

30. Device according to one of claims 1 to 29, characterized in that the one (94) of the two sealing elements (94, 94′) has at least two seal lips (116, 116′) that are engaged for sealing and releasing the slot opening (106).

31. Device according to one of claims 1 to 30, characterized in that another (94′) of the two sealing elements (94, 94′) of the sealing device (88) is shaped in the form of a disk and has a central, approximately circular, recess (118) through which a further instrument, especially a gripping or other cutting instrument, preferably a claw gripper or a myoma drill, can be inserted.

32. Device according to one of claims 1 to 31, characterized in that the central, approximately circular recess (118) of the other (94′) of the two sealing elements (94, 94′) has an internal diameter which is smaller or equal to an outside diameter of the other instrument.

33. Device according to one of claims 1 to 32, characterized in that the other (94′) of the two sealing elements (94, 94′) is arranged in the housing (92) on the longitudinal axis (24) of the main body (14) of the cutting device (12) between the proximal end (26) of the main body (14) and the one (94) of the two sealing elements (94, 94′) especially adjacent to the plane (112) extending to the basic surface (110) of the one (94) of the two sealing elements (94, 94′).

34. Device according to one of claims 1 to 33, characterized in that the at least one, especially the two, sealing element(s) (94, 94′) of the sealing device (88) is/are made of flexible plastic, especially from silicon.

35. Device according to one of claims 1 to 34, characterized in that the cutting elements (20, 20′) of the at least two cutting devices (12, 12′) are essentially chamfered, tapered, conical or sloping, facing outwards away from the distal opening (18, 18′) and/or running inwards towards the distal opening (18, 18′).

36. Device according to one of claims 1 to 35, characterized in that the cutting elements (20, 20′) of the at least two cutting devices (12, 12′) are ground either circumferentially or discontinuously with a smooth edge, wavy edge or toothed edge.

37. Device according to one of claims 1 to 36, characterized in that the cutting elements (20, 20′) of the at least two cutting devices (12, 12′) which are ground with a wavy edge have axially retreating wave valleys and axially advancing wave peaks over the circular arc sections distributed around the circumference.

38. Device according to one of claims 1 to 37, characterized in that the cutting elements (20, 20′) of the at least two cutting devices (12, 12′) which are ground with a toothed edge have circumferentially distributed teeth with axially retreating teeth roots and axially advancing teeth points.

39. Device according to one of claims 1 to 38, characterized in that the drive device (22) is designed as a motor that can be driven electrically, by means of a battery, pneumatically or hydraulically.

40. Device according to one of claims 1 to 39, characterized in that the housing (32) of the gear device (30) and/or the centering and/or guiding element and/or the output drive gearwheel (42) and/or the (two) input drive gearwheel(s) (44, 46) and/or the supporting element (56) and/or the drive bushes (66, 66′) and/or the coupling elements (70, 70′) and/or the sealing device (88) and/or housing element (96) and/or the closing element (98) of the housing (92) and/or the clamping disk (120) is/are made of plastic, especially polyoximethylene, polyester, polyphenylensulphone, ABS, acrylic, polycarbonate, tetrafluorethylene or Impax, duroplastic elastomers, with or without glass fibre reinforcement, or a combination of these.

41. Device according to one of claims 1 to 40, characterized in that the basic bodies (14, 14′) of the at least two cutting devices (12, 12′) and/or the housing (32) of the gear device (30) and/or the centering and/or guiding element and/or the output drive gearwheel (42) and/or the (two) input drive gearwheel(s) (44, 46) and/or the supporting element (56) and/or the drive bushes (66, 66′) and/or the coupling elements (70, 70′) and/or the coupling device (72) and/or the sealing device (88) and/or housing element (96) and/or the closing element (98) of the housing (92) and/or the clamping disk (120) is/are made of metal, especially (stainless) steel, high-grade steel, aluminum, brass, zinc, leaded red brass or an alloy of these.

42. Device according to one of claims 1 to 41, characterized in that the gear device (30) arranged between the at least two cutting device (12, 12′) and the drive device (22) is designed and can be assembled with its individual components in the form of a module.

43. Device according to one of claims 1 to 42, characterized in that the housing (32) of the gear device (30) or the housing element (34) and the housing cover (36) and/or the output drive gearwheel (42) and/or the one or two input drive gearwheel(s) (44, 46) and/or the supporting element (56) and/or the drive bushes (66, 66′) is/are designed and can be assembled in the form of a module.

44. Use of a device according to one of the preceding claims for laparascopical, thoracoscopical or athroscopical and minimum-invasive surgical procedures.
